(19) United States
(12) Patent Application Publication
(10) Pub. No.: US 2026/0207126 A1
(43) Pub. Date: Jul. 23, 2026

(54) AI ALGORITHM HARDWARE ACCELERATOR FOR ADAPTIVE SLEEP MONITORING IN SMART MATTRESSES

(71) Applicant: iFutureLab Inc, Palo Alto, CA (US)

(72) Inventors: Jack Brown, Palo Alto, CA (US); David Harris, Palo Alto, CA (US)

(21) Appl. No.: 19/026,942

(22) Filed: Jan. 17, 2025

(51) Int. Cl. A61B 5/00; G06F 1/3203; G06N 3/092; H03M 1/12

(52) U.S. Cl. CPC A61B 5/4806; G06F 1/3203; G06N 3/092; H03M 1/12

(57) ABSTRACT

An artificial intelligence algorithm hardware accelerator designed for adaptive sleep monitoring in smart mattresses is disclosed. The system includes a data input module that receives and preprocesses physiological data from various sensors, utilizing an analog-to-digital converter and signal normalization circuit. A deep learning acceleration module based on FPGA or ASIC architecture is employed to expedite the computation of deep learning algorithms, such as CNNs and LSTMs, through parallel computing and high-throughput data processing. The strategy generation acceleration module uses reinforcement learning algorithms to optimize mattress adjustment strategies, focusing on parameters like height, softness, hardness, and temperature. A control and adjustment module transmits optimized instructions to mattress adjustment devices with low-latency communication, while a low-power optimization module dynamically manages power supply based on computing load, incorporating a voltage regulator, frequency governor, and power monitor.

### TECHNICAL FIELD

The present disclosure relates to the fields of smart mattresses and artificial intelligence, and more particularly to an artificial intelligence algorithm hardware accelerator for adaptive sleep monitoring and regulation, aimed at enhancing computing efficiency in sleep data analysis, regulation strategy generation, and real-time response in smart mattresses.

### BACKGROUND

The field of smart mattresses and artificial intelligence has seen significant advancements, particularly in the area of sleep monitoring and regulation. Smart mattresses aim to enhance sleep quality by analysing sleep patterns and making real-time adjustments. However, the current systems predominantly rely on software-based algorithms for processing sleep data and generating adjustment strategies. These software solutions often face challenges in terms of computational efficiency, leading to delays in processing complex data and hindering the ability to provide real-time and precise adjustments. Additionally, the high-power consumption of these systems limits their suitability for prolonged use.

Recent developments in artificial intelligence and hardware accelerators, especially in deep learning and real-time data analysis, present potential solutions to these challenges. Hardware accelerators have been shown to significantly improve computing speed and energy efficiency in various applications. Despite these advancements, the integration of such technologies into smart mattress systems remains underexplored. Existing solutions in the market have not fully leveraged the capabilities of hardware accelerators to enhance the performance of adaptive sleep monitoring and regulation.

The limitations of traditional smart mattress systems are evident in their reliance on software algorithms, which result in low computational efficiency and delayed data processing. These systems struggle to achieve real-time and accurate adjustments, which are crucial for effective sleep regulation. Moreover, the high-power consumption associated with these methods poses a barrier to their long-term operation. The current state of technology in this domain lacks a dedicated approach to harness the benefits of hardware accelerators for improving adaptive sleep monitoring.

Given the deficiencies in existing solutions, there is a clear need for an innovative approach that addresses these challenges. Further there is a need to introduce a specialized hardware accelerator. Additionally, there is need to enhance the efficiency of sleep data analysis, strategy generation, and real-time response in smart mattresses, thereby overcoming the limitations of current systems.

### SUMMARY

The present disclosure is described in the following sections by various embodiments. However, it should be understood that the disclosure can be implemented in various forms and is not limited to the specific embodiment provided herein.

Embodiments of the present disclosure provide an artificial intelligence algorithm hardware accelerator for adaptive sleep monitoring and regulation, which comprises a data input module for receiving and preprocessing physiological data from multiple sensors. This module is designed to handle data such as pressure, temperature, humidity, heart rate, and breathing rate, converting these analog signals into digital format through an analog-to-digital converter and performing signal normalization to facilitate subsequent processing by the hardware accelerator. The integration of this module ensures accurate and efficient data acquisition, which is crucial for the real-time monitoring and regulation of sleep conditions.

The advantages offered by this embodiment include enhanced data processing accuracy and efficiency, which are critical for the effective monitoring of physiological parameters. By converting and normalizing sensor data, the system ensures that the subsequent modules receive high-quality input, thereby improving the overall performance of the hardware accelerator in adaptive sleep monitoring applications. Subsequently, the hardware accelerator processes efficient sleep-related data such as pressure, temperature, humidity, heart rate and respiration rate, etc. The hardware accelerator can then be considered as a specialized chip, which improves the efficiency of the AI algorithms in terms of hardware, and also saves power consumption, highlighting the high efficiency, high speed and accuracy as well as the low sub-power consumption.

In accordance with an embodiment of the present disclosure, the deep learning acceleration module is based on Field-Programmable Gate Array (FPGA) or Application-Specific Integrated Circuit architecture (ASIC), which accelerates the calculation of deep learning algorithms, including convolutional neural networks (CNNs) and long short-term memory (LSTM) networks. This module comprises a CNN acceleration unit, a matrix multiplication unit, and an LSTM unit, all of which work in tandem to perform efficient feature extraction and pattern recognition. The use of FPGA or ASIC architecture allows for parallel computing and high-throughput data processing, significantly enhancing the speed and accuracy of deep learning computations.

In accordance with an embodiment of the present disclosure, the strategy generation acceleration module utilizes a Deep Q Network (DQN) or policy gradient algorithm implemented in hardware to optimize the generation of mattress adjustment strategies. This module includes a Q-value calculator, a policy gradient calculation unit, and a reward calculator, which collectively expedite the reinforcement learning process. By employing these algorithms, the module can quickly generate and optimize strategies for adjusting mattress parameters such as height, softness, and temperature, thereby improving the user's sleep quality.

In accordance with an embodiment of the present disclosure, the control and adjustment module comprise an optimized communication interface for low-latency instruction transmission. This module is responsible for sending the optimized adjustment instructions to the multi-functional adjustment device of the mattress, enabling real-time regional adjustments. The low-latency communication interface ensures that the instructions are transmitted swiftly, thereby enhancing the response speed of the regulation device and ensuring timely adjustments to the mattress settings.

In accordance with an embodiment of the present disclosure, the low-power optimization module includes a dynamic power management unit DPMU for controlling power supply based on computing load. This module employs dynamic power management and energy-saving algorithms to reduce the power consumption of the hardware accelerator, making it suitable for long-term operation. The DPMU, along with a voltage regulator and frequency governor, ensures that power is efficiently distributed across the hardware units, thereby minimizing energy consumption while maintaining high performance.

In accordance with an embodiment of the present disclosure, the hardware accelerator further comprises a user interface module for displaying sensor data, adjustment status, and optimization suggestions. This module allows users to manually adjust parameters of the accelerator, providing a user-friendly interface for monitoring and controlling the system. The inclusion of this module enhances the usability of the hardware accelerator, making it accessible to a wider range of users and applications.

### BRIEF DESCRIPTION OF THE DRAWINGS

The accompanying drawings illustrate various embodiments of systems, methods, and embodiments of various other aspects of the disclosure. Any person with ordinary skills in the art will appreciate that the illustrated element boundaries e.g. boxes, groups of boxes, or other shapes in the figures represent one example of the boundaries. It may be that in some examples one element may be designed as multiple elements or that multiple elements may be designed as one element.

In some examples, an element shown as an internal component of one element may be implemented as an external component in another, and vice versa. Furthermore, elements may not be drawn to scale. Non-limiting and non-exhaustive descriptions are described with reference to the following drawings. The components in the figures are not necessarily to scale, emphasis instead being placed upon illustrating principles.

FIG. 1 illustrates a schematic diagram of the overall structure of the hardware accelerator, including modules for deep learning acceleration, data input, strategy generation acceleration, control and adjustment, and low-power optimization, in accordance with an embodiment of the present disclosure;

FIG. 2 illustrates an architecture diagram of the deep learning acceleration module, detailing the hardware implementation of convolutional neural networks and LSTM, including steps for data transmission, buffering, CNN and LSTM processing, matrix multiplication, memory management, sparse matrix optimization, and parallel computing control, in accordance with an embodiment of the present disclosure;

FIG. 3 illustrates a schematic diagram of the hardware structure of the strategy generation acceleration module, showing the hardware acceleration logic of the reinforcement learning algorithm, in accordance with an embodiment of the present disclosure;

FIG. 4 illustrates a schematic diagram of the power management of the low-power optimization module, depicting the mechanism and algorithm of dynamic power management, in accordance with an embodiment of the present disclosure.

It should be noted that the accompanying figure is intended to present illustrations of a few examples of the present disclosure. The figure is not intended to limit the scope of the present disclosure. It should also be noted that the accompanying figure is not necessarily drawn to scale.

### DETAILED DESCRIPTION OF THE EMBODIMENTS

Some embodiments of this disclosure, illustrating all its features, will now be discussed in detail. The words “comprising,” “having,” “containing,” and “including,” and other forms thereof, are intended to be equivalent in meaning and be open ended in that an item or items following any one of these words is not meant to be an exhaustive listing of such item or items, or meant to be limited to only the listed item or items.

It must also be noted that as used herein and in the appended claims, the singular forms “a,” “an,” and “the” include plural references unless the context clearly dictates otherwise. Although any systems and methods similar or equivalent to those described herein can be used in the practice or testing of embodiments of the present disclosure, the preferred, systems and methods are now described.

The present disclosure relates to an artificial intelligence algorithm hardware accelerator specifically designed for adaptive sleep monitoring and regulation in smart mattresses. This hardware accelerator is engineered to enhance the efficiency and responsiveness of sleep monitoring systems by integrating advanced deep learning and reinforcement learning algorithms into a dedicated hardware framework. The disclosure aims to provide a comprehensive solution for real-time data analysis and mattress adjustment, thereby improving the overall sleep quality and comfort for users.

The hardware accelerator comprises several key modules, each serving a distinct function to optimize the performance of the smart mattress system. The data input module is responsible for receiving and preprocessing physiological data from a plurality of sensors, including pressure, temperature, humidity, heart rate, and breathing rate. This module converts analog signals into digital signals and normalizes them for further processing. The deep learning acceleration module, based on Field-Programmable Gate Array (FPGA) or ASIC architecture, accelerates the operation of convolutional neural networks (CNNs) and long short-term memory (LSTM) networks, facilitating efficient feature extraction and pattern recognition. Additionally, the strategy generation acceleration module employs hardware-implemented reinforcement learning algorithms, such as Deep Q Network (DQN) or policy gradient methods, to optimize mattress adjustment strategies.

The control and adjustment module transmits optimized adjustment instructions to the mattress's multi-functional adjustment device, ensuring real-time regional adjustment with low-latency communication. To address power consumption concerns, the low-power optimization module incorporates dynamic power management and energy-saving algorithms, featuring components like a dynamic power management unit DPMU, voltage regulator, and frequency governor. This module is designed to reduce power consumption, making the hardware accelerator suitable for long-term operation.

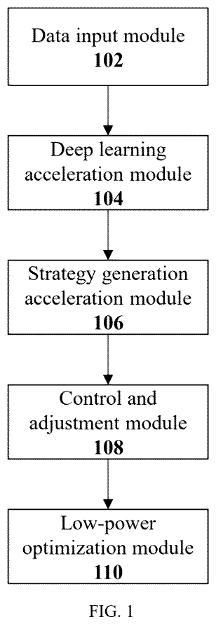

FIG. 1 illustrates a schematic diagram of the overall structure of the hardware accelerator for adaptive sleep monitoring in smart mattresses. The hardware accelerator comprises a data input module 102, a deep learning acceleration module 104, a strategy generation acceleration module 106, a control and adjustment module 108, and a low-power optimization module 110. Each module is designed to perform specific functions that collectively enhance the efficiency and responsiveness of the sleep monitoring system.

The data input module 102 is configured to receive multi-sensor data, including pressure, temperature, humidity, heart rate, and breathing rate. This module converts the analog signals from the sensors into digital signals using an analog-to-digital converter, followed by signal normalization to ensure compatibility with subsequent processing stages. The deep learning acceleration module 104 utilizes FPGA or ASIC architecture to accelerate the computation of deep learning models such as CNNs and LSTMs, enabling rapid feature extraction and pattern recognition from the sensor data.

The strategy generation acceleration module 106 implements reinforcement learning algorithms in hardware to expedite the creation of mattress adjustment strategies. This module is responsible for optimizing parameters such as mattress height, softness, hardness, and temperature, ensuring that adjustments are responsive to the user's physiological state. The control and adjustment module 108 transmits these optimized instructions to the mattress adjustment devices, utilizing an optimized communication interface to minimize latency and enhance response speed.

The low-power optimization module 110 incorporates a dynamic power management unit (DPMU) to manage power consumption efficiently. This module includes components such as a voltage regulator, frequency governor, and power monitor, which work together to adjust power supply based on computing load, thereby ensuring energy-efficient operation of the hardware accelerator.

In one embodiment of the disclosure, the data input module 102 may include an analog-to-digital converter and a signal normalization circuit to enhance the accuracy of sensor data processing. The deep learning acceleration module 104 may employ FPGA or ASIC architecture to achieve parallel computing and high-throughput data processing, facilitating real-time analysis of physiological data.

Additionally, the strategy generation acceleration module 106 may utilize a DQN or policy gradient algorithm implemented in hardware to optimize the generation of mattress adjustment strategies. The control and adjustment module 108 may feature an optimized communication interface for low-latency transmission of adjustment instructions, improving the response speed of the mattress adjustment devices.

In accordance with yet another embodiment, the low-power optimization module 110 may dynamically adjust power consumption using an energy-saving algorithm and DPMU, ensuring continuous operation while minimizing energy usage. This module may also support low-power modes that activate during periods of low computational demand, further enhancing the energy efficiency of the system.

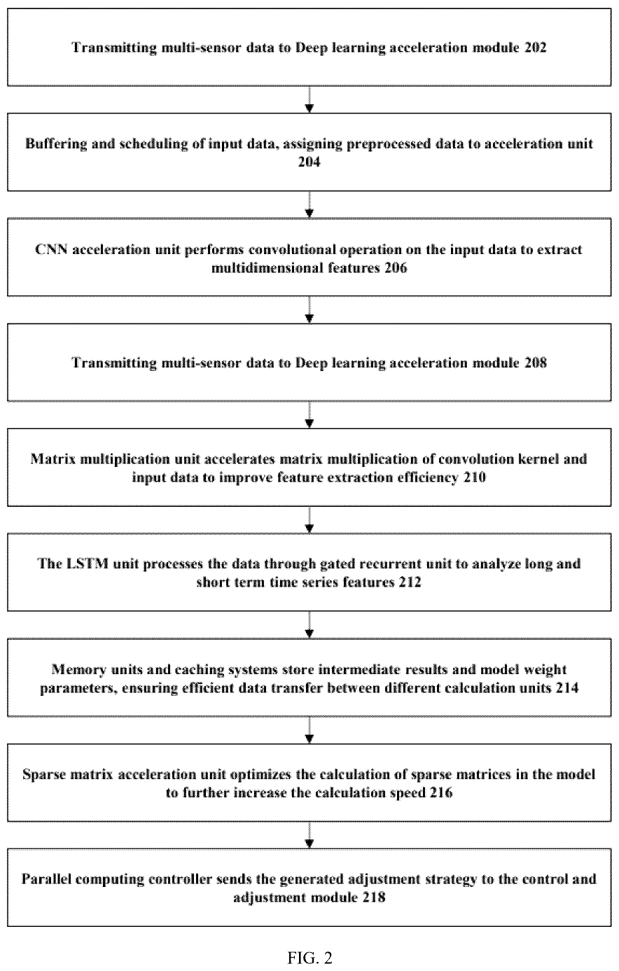

FIG. 2 illustrates the architecture of the deep learning acceleration module, detailing the hardware implementation of convolutional neural networks (CNNs) and long short-term memory (LSTM) networks. This module is integral to the processing of multi-sensor data, enabling efficient feature extraction and pattern recognition. The module begins with step 202, where multi-sensor data is transmitted to the deep learning acceleration module. Step 204 involves buffering and scheduling of input data, assigning pre-processed data to the acceleration unit for further processing.

The CNN acceleration unit, as depicted in step 206, performs convolutional operations on the input data to extract multidimensional features. Further at step 208 transmitting multi-sensor data to Deep learning acceleration module. This is followed by step 210, where the matrix multiplication unit accelerates the multiplication of convolution kernels and input data, enhancing feature extraction efficiency. The LSTM unit, shown in step 212, processes data through a gated recurrent unit to analyse long and short-term time series features, providing a comprehensive understanding of the user's physiological state.

Memory units and caching systems, as indicated in step 214, store intermediate results and model weight parameters, ensuring efficient data transfer between different calculation units. The sparse matrix acceleration unit, in step 216, optimizes the calculation of sparse matrices within the model, further increasing computational speed. Finally, step 218 involves the parallel computing controller sending the generated adjustment strategy to the control and adjustment module, facilitating real-time mattress adjustments.

In one embodiment of the disclosure, the deep learning acceleration module may utilize FPGA or ASIC architecture to support parallel computing and high-throughput data processing. This architecture allows for rapid analysis of physiological data, enabling timely adjustments to the mattress settings. The module may also incorporate adaptive learning capabilities, adjusting model parameters based on long-term physiological data to improve the accuracy of regulation strategies.

Additionally, the memory units and caching systems may be designed to provide low-latency access to stored data, ensuring seamless data flow between processing units. The sparse matrix acceleration unit may employ specialized algorithms to handle sparse data efficiently, reducing computational overhead and enhancing processing speed.

In accordance with yet another embodiment, the parallel computing controller may be configured to prioritize tasks based on the urgency of physiological data, ensuring that critical adjustments are made promptly. This prioritization mechanism enhances the responsiveness of the system, providing users with a more adaptive sleep environment.

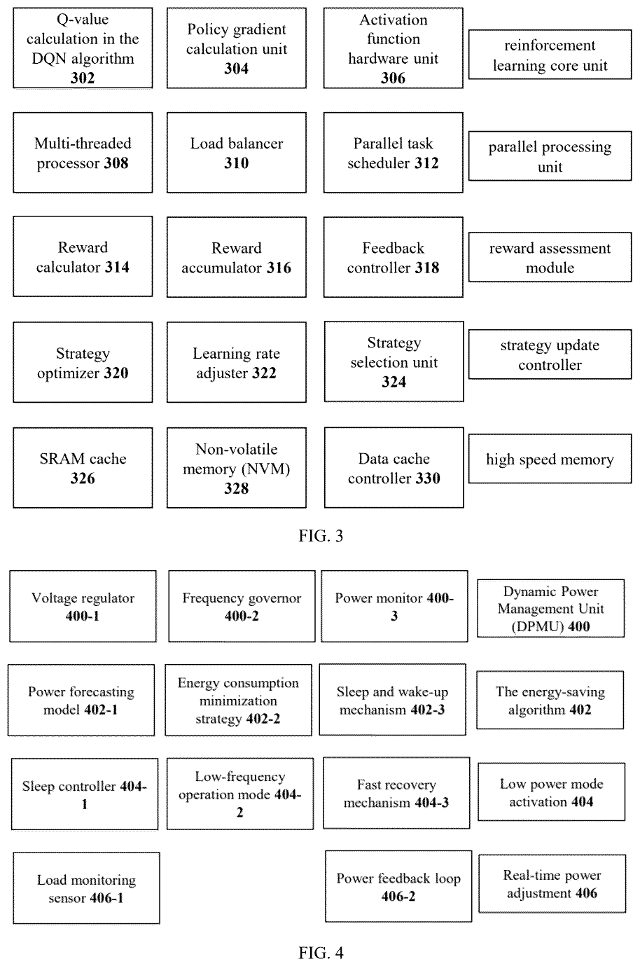

FIG. 3 illustrates the hardware structure of the strategy generation acceleration module, showcasing the hardware acceleration logic of the reinforcement learning algorithm. This module is pivotal in generating optimized mattress adjustment strategies by employing various components that work in tandem to enhance computational efficiency and responsiveness.

The Q-value calculator 302 is utilized for updating Q-values in the DQN algorithm, determining the optimal action for each state. The policy gradient calculation unit 304 generates adjustment strategies in continuous space, accommodating diverse mattress adjustment requirements. The activation function hardware unit 306 accelerates the computation of activation functions such as ReLU and Sigmoid, thereby expediting strategy optimization.

The multi-threaded processor 308 supports simultaneous execution of multiple computing threads, facilitating rapid multi-path strategy computation. The load balancer 310 dynamically allocates computing tasks by monitoring processor loads in real-time, ensuring efficient hardware resource utilization. The parallel task scheduler 312 assigns tasks to processing units based on the priority of physiological data, enabling swift strategy evaluation.

In one embodiment of the disclosure, the strategy generation acceleration module may implement a reward calculator 314 to assess the reward value of adjustment strategies based on sleep improvement indicators like deep sleep ratio and spinal support. The reward accumulator 316 aggregates rewards during the adjustment process, estimating the long-term impact of strategies. The feedback controller 318 relays reward results to the reinforcement learning core unit for strategy updates.

Additionally, the strategy optimizer 320 updates strategy parameters using gradient descent or ascent methods, refining adjustment accuracy. The learning rate adjuster 322 dynamically modifies the learning rate based on feedback stability and reward trends, accelerating strategy convergence or preventing over-adjustment. The strategy selection unit 324 chooses the strategy with the highest reward value from alternatives, transmitting it to the control and regulation module.

In accordance with yet another embodiment, the SRAM cache 326 may store temporary calculation results and model weight parameters, providing low-latency access. The non-volatile memory (NVM) 328 stores adjustment strategies and user physiological data history, supporting long-term model optimization. The data cache controller 330 manages data read and write operations, enhancing transmission efficiency and storage performance.

The strategy generation acceleration module may also incorporate a high-speed memory interface to facilitate rapid data exchange between components, further optimizing the reinforcement learning process. This configuration ensures that the system can adapt to changing user needs and environmental conditions, providing a personalized sleep experience.

FIG. 4 illustrates the dynamic power management unit (DPMU) within the low-power optimization module, which is crucial for managing the power consumption of the hardware accelerator. The DPMU 400 dynamically adjusts the power supply to various hardware components based on the computational load, ensuring energy-efficient operation. This unit employs sophisticated power management strategies to independently control the power supply for each hardware unit, thereby enhancing energy efficiency.

The voltage regulator 400-1 adjusts the voltage output according to real-time load conditions, providing sufficient voltage for high performance during peak loads and reducing voltage to conserve energy during low loads. The frequency governor 400-2 modulates the clock frequency based on computing demand, allowing resources to operate at high frequencies when necessary and reducing frequencies to save power during low demand periods. The power monitor 400-3 continuously tracks the power consumption and operational status of each hardware unit, serving as a feedback source for power regulation.

In one embodiment of the disclosure, the energy-saving algorithm 402 collaborates with the DPMU to intelligently control power allocation and optimize power usage strategies. The power forecasting model 402-1 predicts future power requirements based on historical computing load and data processing needs, allowing for proactive power adjustments. The energy consumption minimization strategy 402-2 optimizes task scheduling and resource allocation to balance the load across different hardware units, minimizing total energy consumption.

The sleep and wake-up mechanism 402-3 places hardware units into sleep mode during prolonged low-load states and quickly reactivates them when needed. The sleep controller 404-1 manages this process, ensuring that idle units are efficiently put to sleep. The low-frequency operation mode 404-2 activates when the overall load is low, further reducing energy consumption. The fast recovery mechanism 404-3 ensures a swift return to high-performance states when required, avoiding long delays.

The real-time power adjustment mechanism 406 involves collaboration between the DPMU and energy-saving algorithms, ensuring rapid response to changes in computational load. The load monitoring sensor 406-1 detects the workload of each hardware unit in real time, while the power feedback loop 406-2 quickly adjusts power distribution and voltage output based on feedback signals from load sensors.

The low-power optimization module may also support various low-power modes that automatically activate during periods of reduced computational demand, further enhancing the system's energy efficiency. This configuration ensures that the hardware accelerator can maintain high performance while minimizing energy consumption, providing a sustainable solution for long-term operation in smart mattresses.

Although, the present disclosure has been described with reference to certain preferred embodiments and examples thereof, other embodiments and equivalents are possible. Even though numerous characteristics and advantages of the present disclosure have been set forth in the foregoing description, together with functional and procedural details, the disclosure is illustrative only, and changes may be made in detail, within the principles of the disclosure to the full extent indicated by the broad general meaning of the terms. Thus, various modifications are possible of the presently disclosed system and process without deviating from the intended scope and spirit of the present disclosure.

What is claimed is:

1. An artificial intelligence algorithm hardware accelerator for adaptive sleep monitoring in smart mattresses, comprising:
a data input module configured to receive and preprocess physiological data from a plurality of sensors, wherein the data input module comprises an analog-to-digital converter and a signal normalization circuit for converting and normalizing the sensor data;
a deep learning acceleration module based on FPGA or ASIC architecture, configured to accelerate the computation of deep learning algorithms, comprising convolutional neural networks (CNNs) and long short-term memory (LSTM) networks, wherein the deep learning acceleration module performs parallel computing and high-throughput data processing;
a strategy generation acceleration module utilizing reinforcement learning algorithms, comprising Deep Q Network (DQN) or policy gradient, configured to generate mattress adjustment strategies by optimizing parameters related to mattress height, softness, hardness, and temperature;
a control and adjustment module configured to transmit optimized adjustment instructions to mattress adjustment devices, wherein the control and adjustment module features an optimized communication interface for low-latency instruction transmission; and
a low-power optimization module comprising a dynamic power management unit (DPMU) configured to adjust power supply based on computing load, wherein the low-power optimization module comprises a voltage regulator, frequency governor, and power monitor for dynamic power management;
wherein the hardware accelerator is configured to process and analyze physiological data in real-time, enabling adaptive sleep monitoring and regulation.

2. The hardware accelerator of claim 1, wherein the data input module further comprises an analog-to-digital converter and a signal normalization for improving the processing accuracy of sensor data.

3. The hardware accelerator of claim 1, wherein the deep learning acceleration module further comprises a CNN acceleration unit configured to perform convolutional operations on input data to extract multidimensional features, and an LSTM unit configured to process data through a gated recurrent unit for analyzing long and short-term time series features.

4. The hardware accelerator of claim 3, wherein the deep learning acceleration module further comprises memory units and caching systems configured to store intermediate results and model weight parameters, ensuring efficient data transfer between different calculation units.

5. The hardware accelerator of claim 1, wherein the strategy generation acceleration module further comprises a Q-value calculator configured to update Q-values in the DQN algorithm, and a policy gradient calculation unit configured to generate adjustment strategies in continuous space.

6. The hardware accelerator of claim 5, wherein the strategy generation acceleration module further comprises an activation function hardware unit configured to accelerate the calculation of activation functions, and a multi-threaded processor configured to support simultaneous multi-thread computing.

7. The hardware accelerator of claim 1, wherein the control and adjustment module is based on FPGA core architecture and is configured to ensure precise adjustment of the mattress area.

8. The hardware accelerator of claim 1, wherein the low-power optimization module further comprises an energy-saving algorithm configured to optimize power allocation and employ strategies like sleep and wake-up mechanisms to minimize energy consumption.

9. The hardware accelerator of claim 1, further comprising a user interface module configured to visually display sensor data, adjustment status, and optimization suggestions, allowing users to manually adjust parameters of the accelerator.

10. A method for adaptive sleep monitoring and regulation using the artificial intelligence algorithm hardware accelerator of claim 1, comprising:
receiving and preprocessing physiological data from a plurality of sensors using the data input module;
accelerating the calculation of deep learning algorithms for sleep data analysis using the deep learning acceleration module;
generating mattress adjustment strategies through accelerated reinforcement learning algorithms using the strategy generation acceleration module;
transmitting the generated strategies to mattress adjustment devices using the control and adjustment module for real-time regulation; and
dynamically managing the power consumption of the hardware accelerator using the low-power optimization module.

10. The method of claim 10, wherein the preprocessing comprises converting analog signals to digital signals and normalizing the digital signals.

11. The method of claim 10, wherein the accelerating deep learning algorithm calculations comprises using a hardware architecture selected from the group consisting of FPGA and ASIC.

12. The method of claim 10, wherein the generating mattress adjustment strategies comprises using a DQN or policy gradient algorithm.

13. The method of claim 10, wherein the transmitting real-time adjustment instructions is facilitated by an optimized communication interface.

14. The method of claim 10, wherein the dynamically managing power consumption comprises employing a dynamic power management algorithm to adjust power levels based on computational demand.
